(19) United States
(12) Patent Application Publication
(10) Pub. No.: US 2003/0097079 A1
(43) Pub. Date: May 22, 2003

(54) Biopsy needle sheath

(76) Inventor: Maurice M. Garcia, New Orleans, LA (US)

Correspondence Address: Colin P. Abrahams, Suite 400, 5850 Canoga Avenue, Woodland Hills, CA, 91367, US

(21) Appl. No.: 10/273,452

(22) Filed: Oct. 18, 2002

Related U.S. Application Data
(60) Provisional application No. 60/348,466, filed on Oct. 19, 2001.

(51) Int. Cl. A61B 10/00

(52) U.S. Cl. 600/567

(57) ABSTRACT

A sheath for a biopsy needle for removing a sample from a living body, the sheath comprises a substantially tubular structure configured to accommodate at least a portion of a biopsy needle. The tubular structure has a side wall, a first end opening which in use will remain outside of the body, and a second end opening which will in use insert into the body. The tubular portion has a length between the first and second openings which is greater than the distance between sample to be removed and the outside of the body.

### CROSS REFERENCE TO RELATED APPLICATION

This application claims the benefit of U.S. Provisional Patent Application No. 60/348,466 filed Oct. 19, 2001, the contents of which are incorporated by reference herein in their entirety.

### FIELD AND BACKGROUND OF THE INVENTION

This invention relates to accessory devices for use with biopsy needles, and more particularly such accessory devices when used in conjunction with biopsy needles during the biopsy procedure, whereby the accessory, in various manners, is able to control the amount of bleeding resulting from such biopsy. Furthermore, such accessory devices can minimize the risk of, upon withdrawal of the biopsy needle, seeding the biopsy needle's tract (through extra-tumor subcutaneous tissue planes) with biopsy specimen cells. Preferably, the biopsy needle accessory at least reduces the amount of bleeding resulting from the biopsy, and may be sufficiently effective so as to eliminate such bleeding entirely.

A biopsy has become a standard tool used by health professionals in the course of medical treatment. During the course of a biopsy procedure, a doctor removes a designated piece of tissue from a living body for the purposes of analyzing the tissue and diagnosing the medical condition which require treatment. A Needle-Biopsy, or removal of a piece of tissue from the human body using a ‘biopsy needle’, is typically carried out by inserting a needle into the body, by puncturing the skin and further inserting the needle until it penetrates the specific organ or portion thereof, the condition of which is being analyzed. For example, biopsies may be taken of lung, liver, kidney and other internal organs. A site will be selected for insertion of the needle, and medical practitioners, through experience and with the aid of Radiologic imaging modalities, are able to assess, according to the amount of needle inserted and the depth thereof in the body, that the tissue required has been penetrated. At this point, standard biopsy needles extract the tissue from the organ, and the needle, containing the extracted tissues for analysis is withdrawn from the body.

A biopsy removes tissue from the body, and in so doing, leaves a space or “tract” where the removed tissue used to be. This results in bleeding and blood loss around the tract.

A significant and potentially deleterious problem associated with the taking of biopsies is bleeding, and related sequellae such as irritative pain, infection, life-threatening blood loss, and direct organ damage/loss from bleeding. Such post needle biopsy complications are relatively uncommon, but do occur with sufficient frequency, morbidity and mortality that many medical centers do admit all post biopsy patients overnight for observation. Risk of bleeding related complications necessitates the expenditure of vast resources, time and money. Any product that minimizes such risk would serve to benefit, first and foremost, those patients who each year suffer procedure related complications. In addition, it would eliminate unnecessary inconvenience to patients, and the great cost of resources and money to our health care system. While bleeding may, to some extent, occur at the point where the needle penetrates the skin and subcutaneous tissue planes, more significant bleeding is likely to occur at that point in the selected organ from which tissue has been removed.

Needle biopsy presents another kind of problem to which the aforementioned designs may be applicable. Specifically, recent studies have shown that needle biopsy of malignant tumor tissue can be associated with seeding of the cancerous cells to tissues that lie well outside of the vicinity of the tissue mass being biopsied (i.e. tissues lying along the trajectory of the biopsy needle from skin surface to target tissue). Such seeding occurs when tumor cells are dislodged from the circumscribed tumor mass by the entry of the biopsy needle. When the biopsy needle is withdrawn, a few such cells adhere to the biopsy needle and are “dragged” out of the tumor and thus “seeded” to tissues outside of the tumor's area including, the needle tissue tract, local lymphatic channels and lymph nodes by lymphatic drainage, and into the vascular system by hematogeous local venous drainage. The frequency with which such seeding occurs is relatively low, but its occurrence is associated with a tremendous cost to the patient. It may mean that a previous presumably curative surgery has been rendered palliative only. Furthermore, re-imaging, repeat study work-up, repeat chemotherapy, and of course re-operation can each and all carry with them a significant risk of morbidity and mortality. The latter presumes that the seeding of the tract was (1) detected by the physician, and (2) detected early enough for useful intervention. The worst possible outcome is that such seeding goes undetected until after the window of opportunity for intervention has passed. Also, as mentioned, the frequency of such seeding is low enough that perhaps some physicians could not be sufficiently vigilant after biopsy to detect such seeding.

### SUMMARY AND DESCRIPTION OF THE INVENTION

The present invention is directed towards a biopsy needle accessory, preferably in the form of a sheath or tube, for use in conjunction with the biopsy needle during the biopsy procedure when the tissue from the organ is extracted. The invention has the effect whereby the biopsy needle's wound or tract may be plugged, cauterized or otherwise treated to reduce or eliminate bleeding resulting from the biopsy, and the consequential problems associated with such bleeding within either or both the target organ biopsy tract as well as within any of the subcutaneous tissue planes traversed by the biopsy needle as it reaches the target organ.

According to one aspect of the invention, the accessory for use with a biopsy needle comprises a sheath or tube which surrounds the biopsy needle, but is able to slide up and down the biopsy needle. In one form, the sheath or tube accessory of the invention is placed against the skin, together with the biopsy needle. The biopsy needle is then inserted so as to extend into the organ from which tissue is being extracted. Initially, only the biopsy needle, under Radiologic guidance, is advanced to the surface of the target organ. Afterward, the accessory sheath (Third Sheath or Plug/Advancing Sheath) is advanced to the surface of the organ. Then the Biopsy Needle is discharged into the target organ. The accessory sheath (Third Sheath or Plug/Advancing Sheath) is then fully advanced into the fresh biopsy tract within the target organ.

While so inserted, the accessory sheath of the invention may operate in several ways. According to one embodiment of the invention, the biopsy needle sheath of the invention may comprise a detachable portion which remains in the organ from which the tissue has been extracted. Upon removal of the biopsy needle and needle sheath, a removable component of the accessory sheath remains in the tissue, operating as a hemostatic cylinder or plug, and thereby preventing or reducing bleeding of the tissue (hemostasis), since the plug effectively provides a seal to prevent such bleeding by two methods: (1) The body of the Plug directly tamponnades the bleeding tract tissue surface; (2) the Plug is made of a material that is itself hemostatic and fully absorbable.

For use in the scenario of prevention of tumor seeding, the same Advancing Sheath/Plug design can be used. In this case, only the Plug ever makes contact with the tumor cells, and it remains in place within the tumor area after the needle is withdrawn. Most importantly, the Plug and the Advancing Sheath, together, provide a barrier between the tumor-contaminated biopsy needle and the non-tumor containing tissue lying along the needle trajectory from skin to tumor. In this way, withdrawal of the potentially contaminated biopsy needle can be made a “clean” procedure.

In another form, the biopsy needle sheath of the invention may be connected to a source as will be described, and, upon activation of the source, the wound and bleeding created by the removal of the tissue from the organ may be cauterized, frozen or otherwise treated to prevent bleeding. For example, the biopsy needle sheath of the invention may be connectable to an electrical source, and upon application of an appropriate amount of power, that portion of the biopsy needle sheath embedded within the tissue will cauterize the tissue surrounding it to prevent or eliminate bleeding. In an alternative form, a “cryo-source” may be applied to the sheath of the invention, dropping the temperature of at least an effective portion of the biopsy needle sheath so as to freeze the wound or tract and bleeding formed by removal of the tissue. In both cases, non-bleeding and/or interspersed vital tissues are protected from either the applied cautery or cryogenic application by a layer of electrical/or cryogenic insulation coating along a portion of the sheath's length.

According to another important aspect of the invention, there is provided a biopsy needle sheath device which may also comprise a delivery system. In many instances where needle biopsies are used, including minimally invasive surgical procedures where a narrow device with a long axis (such as any type of needle, or other such solid or hollow tube) is made to penetrate the body, it can be useful to use the preferably bioabsorbable sheath device of the invention as a delivery vehicle for the introduction of a variety of materials, such as: radio-opaque markers, dyes, drug agents, chemical agents, wires and coils for radio-frequency local therapy, etc. The sheath of the invention may therefore be capable of serving as a platform for the simultaneous delivery of materials, by, for example, embedding these materials within the thickness of the bioabsorbable sheath.

The delivery system application using, for example, a bioabsorbable sheath device in accordance with the invention permits a one way introduction of the “vehicle” over the needle (or the long axis of the surgical device being introduced into the patient). There is often a need to implant something deep within the body, while using a minimally invasive procedure, by gaining access to the target site with a hollow needle, and then advancing some material to the site, through the lumen of the hollow member. Such a requirement would normally demand a two step procedure: one for the biopsy and withdrawal of the lumen-occupying biopsy needle and specimen, and another for the introduction and “packing” of the material into the tissue site. With a single channel, a two step procedure is required. The device of the invention allows “two way traffic” (withdrawal of material from the patient or biopsy, while advancing a separate material into the patient (radio-opaque marker, dyes, etc) in a single step procedure, because separate withdrawal and delivery processes are occurring in different places. Withdrawal is through the biopsy needle lumen, and delivery over the biopsy needle.

The importance of rendering an otherwise two-step process into a single step cannot be underscored. For example, to leave a radio-opaque marker at the exact site of a tiny tumor being biopsied, the current technology demands that the (inner) biopsy needle component (containing the specimen) be withdrawn first before the marker is introduced. The problem is that such a sequence of events is cumbersome, and, if by chance, the hollow (over) needle is displaced even slightly as the inner biopsy needle/specimen is being withdrawn, then, when the marker is laid into place, the marker will not be advanced to the exact site as from where the biopsy was taken. The disconnect can make all the difference if the “marker” is to be used to guide radiation, or re-biopsy, or needle delivery of a medication or radiation treatment, etc. In short, to the extent that needle biopsy results direct treatment or diagnostic follow-up, marking the exact site of biopsy can be vitally important. Any spatial disconnect between the true site of biopsy and the where the marking or treatment material is laid in place at the time of biopsy, may lower the specificity of the biopsy procedure. The invention, by virtue of allowing simultaneous withdrawal of specimen while introducing a marker or material for future treatment at the exact site of biopsy, is a tremendous and novel solution to this problem, and increases the specificity of whatever treatment or marking is being made possible by the biopsy procedure.

The lumen of a hollow needle will allow passage of many materials deep into the body. The problem is that one never really knows when the material(s) have been sufficiently advanced into the body so that they lie at the target site. Also, since the materials are advanced “blindly” through the needle lumen, one never knows if they have been deformed (e.g. a delicate wire with fine ends/terminals) during passage.

The invention, however, may allow one to be much more certain where and when the bioabsorbable sheath (and its contents) have been advanced to the very tip of the biopsy needle (i.e. maximum specificity), because all of the relevant lengths are known entities. This includes the length of the biopsy needle below the skin surface, known by virtue of proposed hatch-marks along the biopsy needle, and the lengths of the “Advancing Sheath” and “Bioabsorbable Sheath” by proposed hatch-marks along the latter. For example, the biopsy needle and Advancing Sheath can have a colored arrow at the proximal-most end of each, such that when these arrows are approximated, the advancing sheath will have been advanced to the exact tip of the biopsy needle tip, as desired. Such near certainty may be possible with the current industry standard if what is advanced is totally rigid. If it is flexible in any way, the operator will never know if it has bent/coiled within the hollow lumen, hence making any length markers unreliable.

To facilitate the above delivery/withdrawal duality, the biopsy sheath of the invention may incorporate radio-opaque markers (which are also visible to the naked eye) which can be placed on, for example, the tip and shaft of the “Cauterizing Sheath” device and the “Advancing Sheath” of the “bioabsorbable sheath” device described herein, as well as the biopsy needle over sheath.

Various aspects of the invention therefore relate to devices which can address: hemorrhage associated with a needle biopsy; tumor seeding associated with needle biopsy; and delivery devices for myriad materials which can be embedded within the bioabsorbable sheath.

### BRIEF DESCRIPTION OF THE DRAWINGS

FIGS. 1, 2 and 3 show schematic representations of sequential steps in the use of a biopsy needle and sheath of the invention, illustrating cauterization of tissue;

FIGS. 4, 5 and 6 illustrates a different embodiment of the sheath of the invention;

FIGS. 7 to 10 show yet a further embodiment of the sheath of the invention;

FIGS. 11 and 12 show a needle and sheath of the invention and sequential steps of the operation and effect thereof;

FIGS. 13 and 14 show a different embodiment of a needle and sheath of the invention and sequential steps of the operation and effect thereof;

FIG. 15 illustrates the process of tumor seeding in conventional biopsy systems;

FIGS. 16 and 17 shows a variation of the sheath of the invention when used with a biopsy needle to reduce or eliminate the effect of tumor seeding; and

FIG. 18 shows a series of different types of bioabsorbable plugs in accordance with the intention

### DESCRIPTION OF DRAWINGS AND PREFERRED EMBODIMENTS

The invention will now be described with reference to the accompanying drawings.

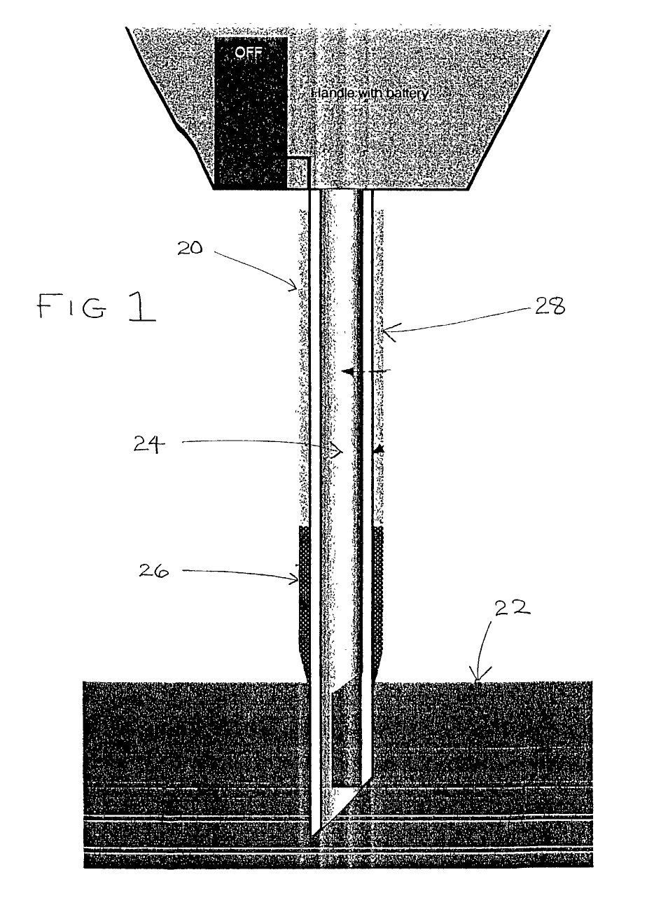

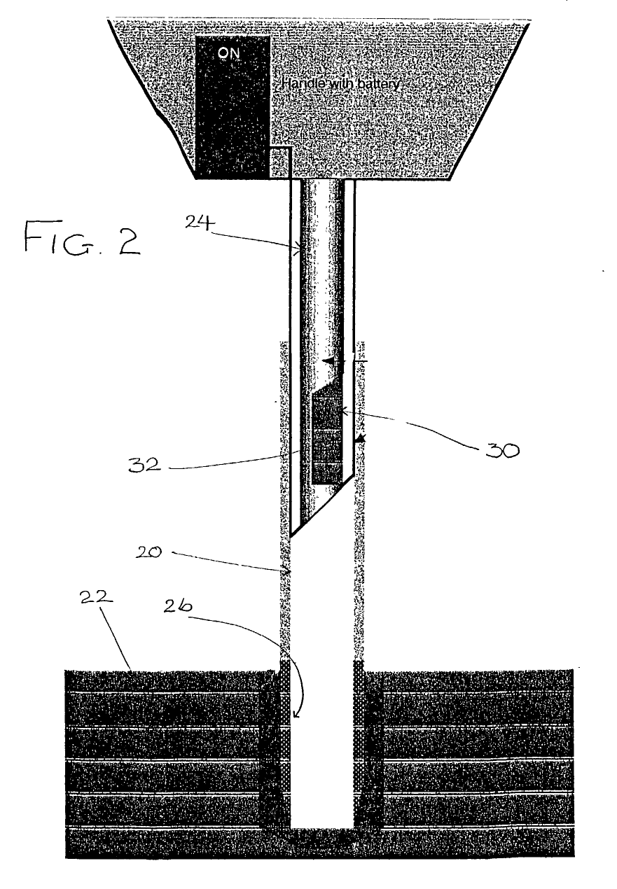

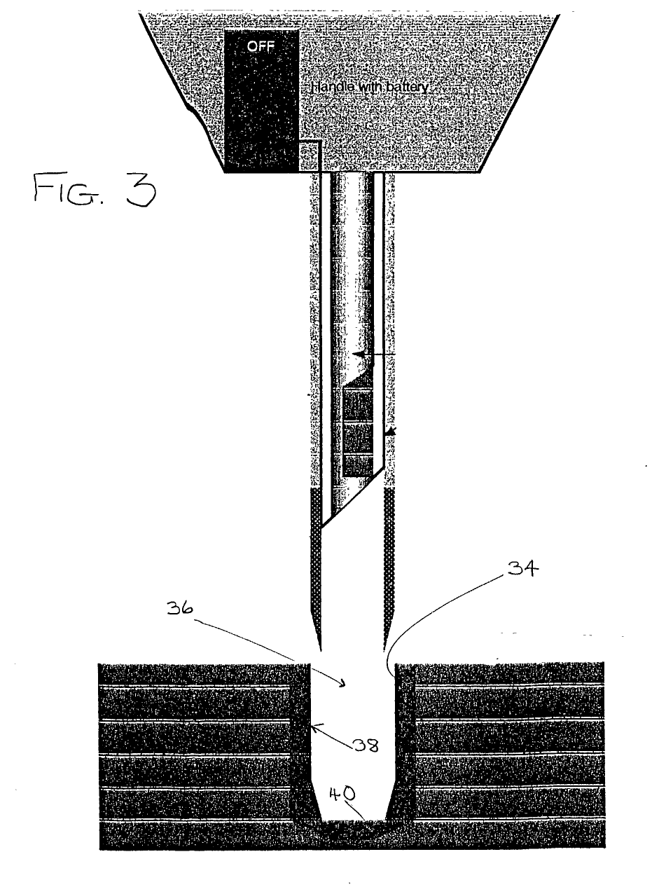

FIGS. 1, 2, and 3 show an embodiment of a biopsy needle sheath 20 of the invention when used to cauterize the tissue 22 of the organ from which the tissue sample has been removed. FIG. 1 shows the biopsy needle 24 including needle introducer sheath 26, embedded in the tissue 22, which may be lung, liver or other living tissue of an organ from which a biopsy is being taken. In FIG. 1 there is shown the biopsy needle sheath 20, including a cauterizing segment 26. The needle 24 is located within the tissue 22, but in FIG. 1, the needle sheath 20 still remains outside of the tissue 22. The insulated sheath 20 comprises a regular portion 28 which may be comprised of metal or other material, and a cauterizing segment 26. In FIG. 2, the insulated sheath 20 has been advanced so that it is now in the tissue 22, and the biopsy needle 24, with the tissue sample 30 contained within the recess 32 thereof, is being withdrawn from the sheath 20. The cauterizing segment 26 of the sheath 20 is in the tissue 22. The cauterizing segment 26 of the sheath 20 is activated, thereby cauterizing the tissue surrounding or forming the tract from which the tissue sample has been removed, forming cauterized tissue 34. Thereafter, as shown in FIG. 3, both biopsy needle 24, with tissue sample 30, as well as the insulated sheath 20, are withdrawn, and the tract 36 from which the tissue 30 has been removed has cauterized walls 38 and a base 40, as a result of the power applied to the sheath 20. In this way, bleeding of the tissue 22 as a consequence of the extraction of the tissue sample 30 is significantly minimized, and may even be reduced to the point where it no longer exists.

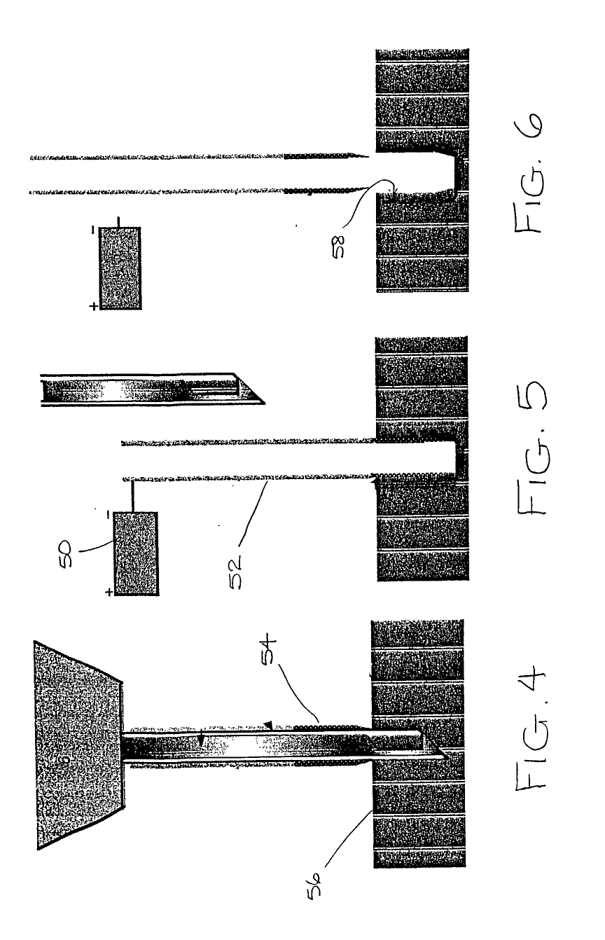

FIGS. 4, 5 and 6 show different embodiments of similar procedures illustrated in FIGS. 1, 2, and 3. In FIG. 5, there is illustrated a power source 50, connected to the insulating sheath 52, so that the cauterizing segment 54 will heat up and initiate cautery of the raw tissue 56 within the organ. Once this has occurred, as illustrated in FIG. 6, and cautery has been completed, the insulating sheath 52 and the cauterizing segment 54 are withdrawn. It will be seen from the darkened portion 58 of the tissue 56 that the tissue has been appropriately cauterized to minimize or even eliminate bleeding.

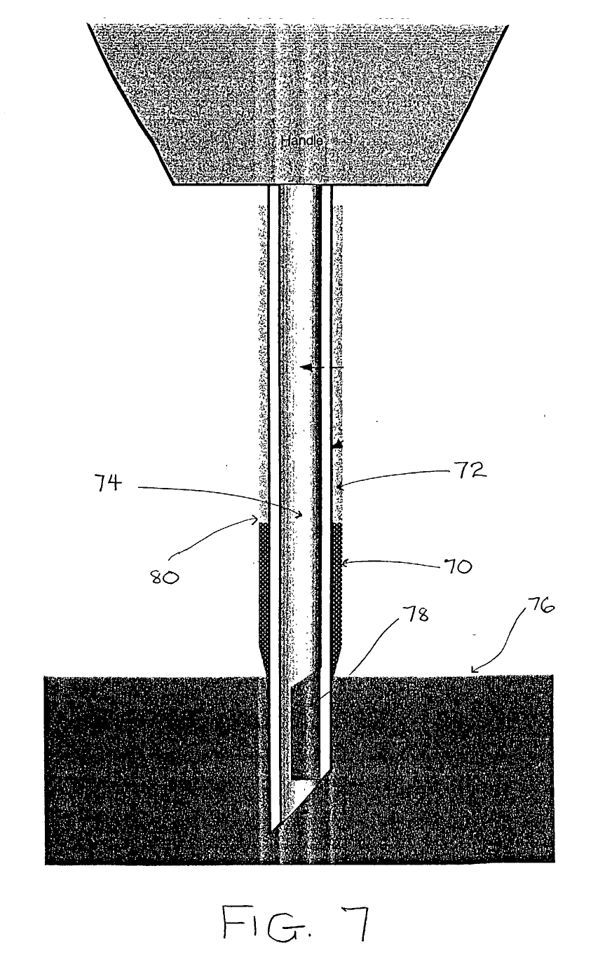

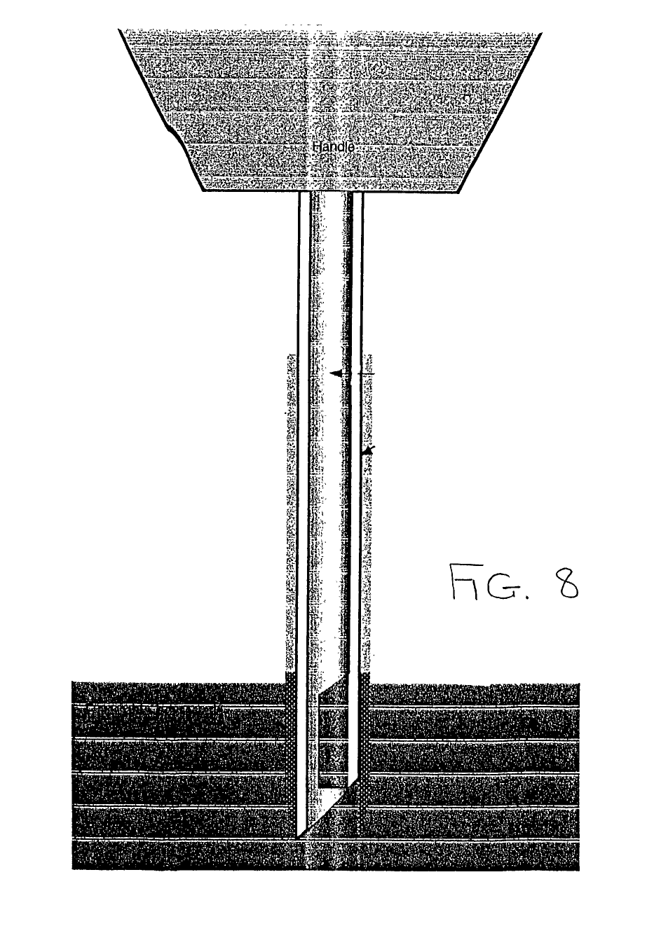

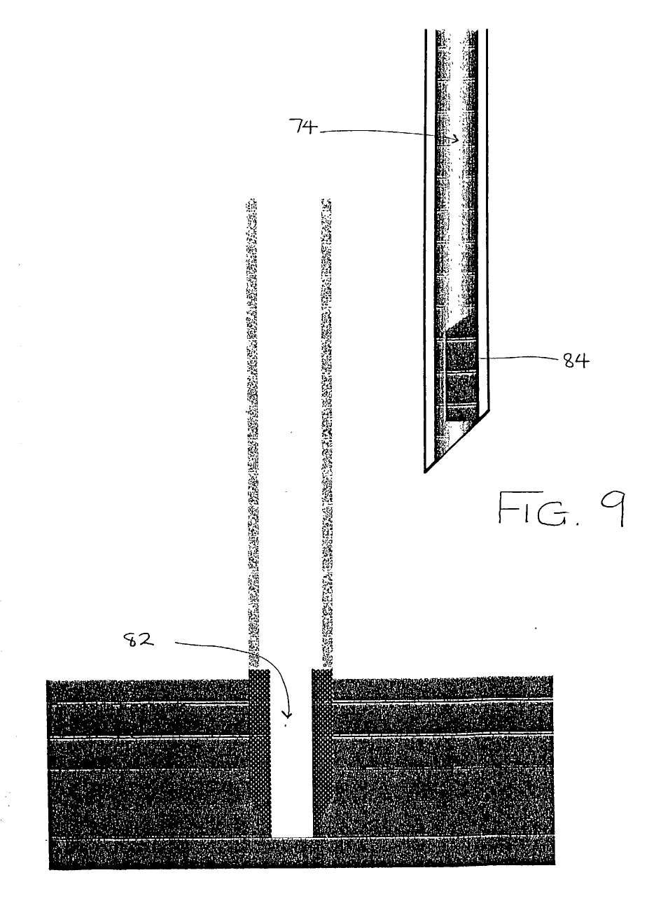

FIGS. 7 to 10 show an alternative embodiment of the invention. Instead of the raw tissue within the biopsy organ being cauterized, a hemostatic plug 70 is inserted using the biopsy needle sheath 72 of the invention, and this, too, has the effect of limiting of eliminating bleeding which results from the removal of the biopsy tissue sample.

In FIG. 7, there is shown a biopsy needle 74 having the needle introducer sheath 72, which is in turn surrounded by the stabilizing cylinder. The biopsy needle 74 and introducer sheath 72 is shown penetrating the tissue 76 of the organ from which the biopsy sample 78 is being taken. The stabilizing cylinder or biopsy needle sheath 72 has one end 80 just touching the tissue 76 from which the biopsy has been taken. That end 80 of the biopsy sheath 72 or stabilizing cylinder includes a detachable hemostatic cylinder 70, as indicated in the drawings.

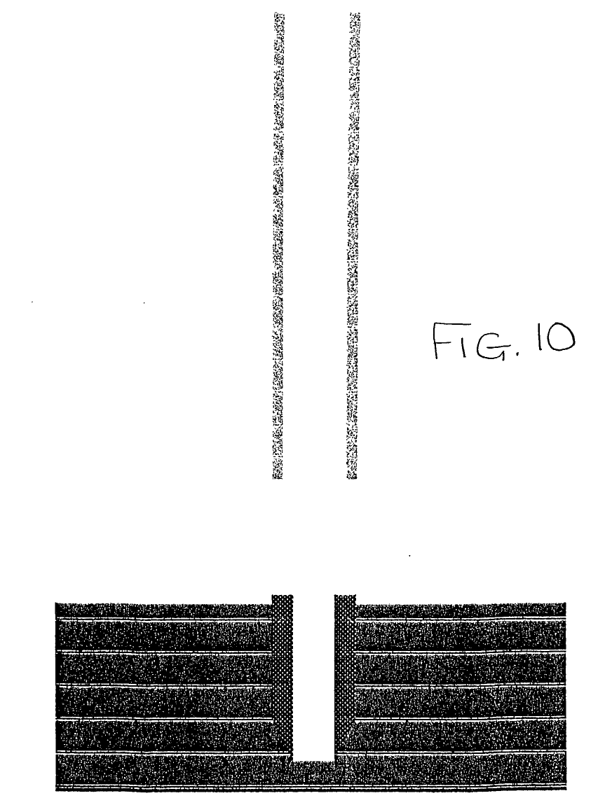

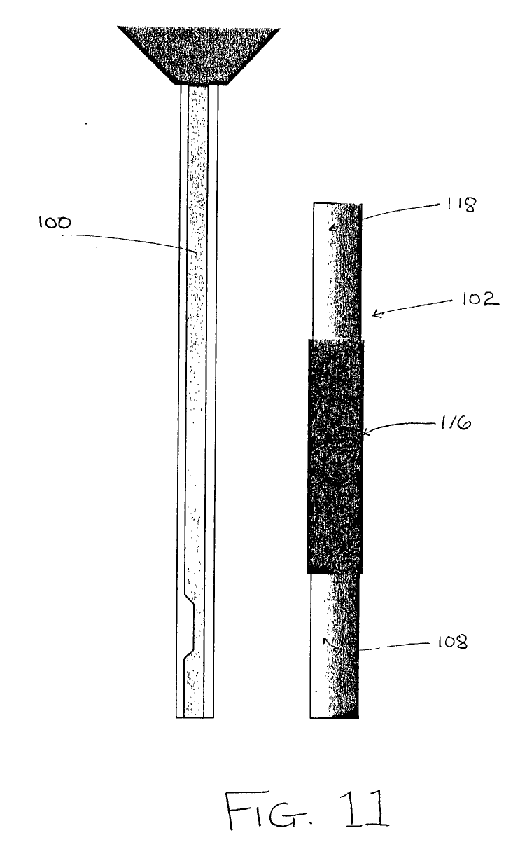

As shown in FIG. 8, the stabilizing cylinder or sheath 72 is advanced into the tissue, and in FIG. 9 it is shown that the biopsy needle 74 has been removed. However, the stabilizing cylinder 72 remains in the tissue 76, with the detachable hemostatic cylinder 70 within the tract 82 formed by the removed tissue 84. FIG. 10 shows that the remainder of the stabilizing cylinder 72 is detached from the hemostatic cylinder 70, leaving the hemostatic cylinder 70 within the tissue 76, which thereby forms a plug to prevent or eliminate bleeding.

As an alternative, or in addition, to having a detachable hemostatic cylinder 70, a hemostatic plug can also be inserted through the cylinder, and this will have essentially the same effect as the hemostatic cylinder indicated in FIGS. 7-10.

The nature of the hemostatic cylinder and hemostatic plug, the materials, as well as the dimensions thereof will be discussed below.

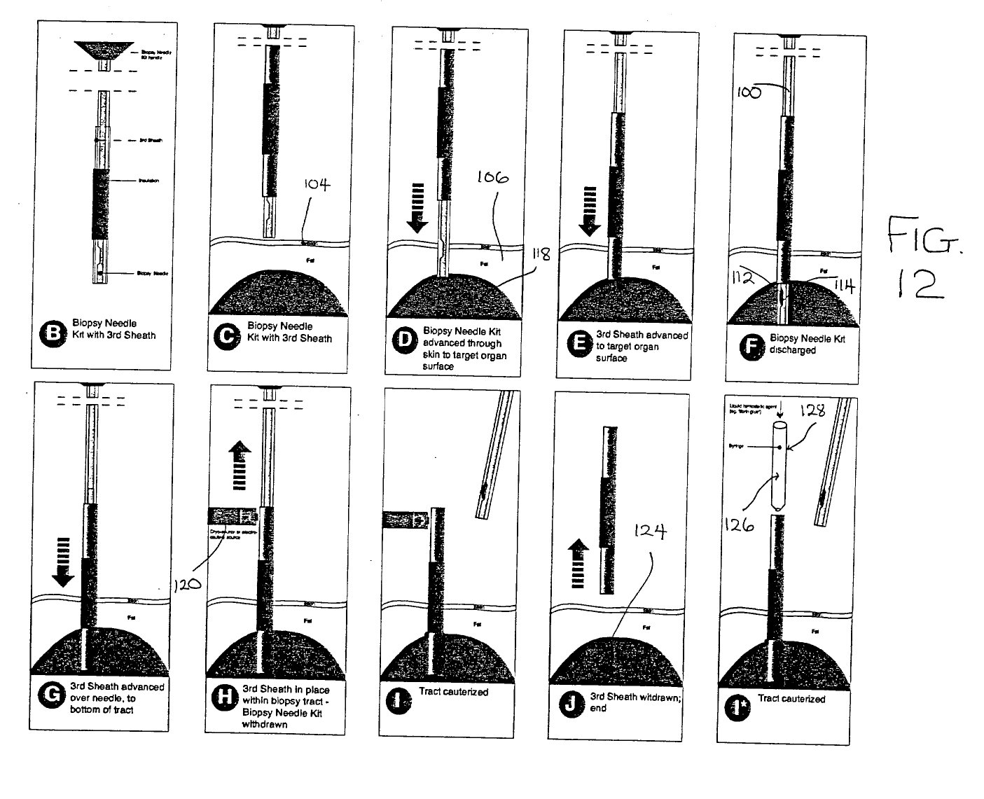

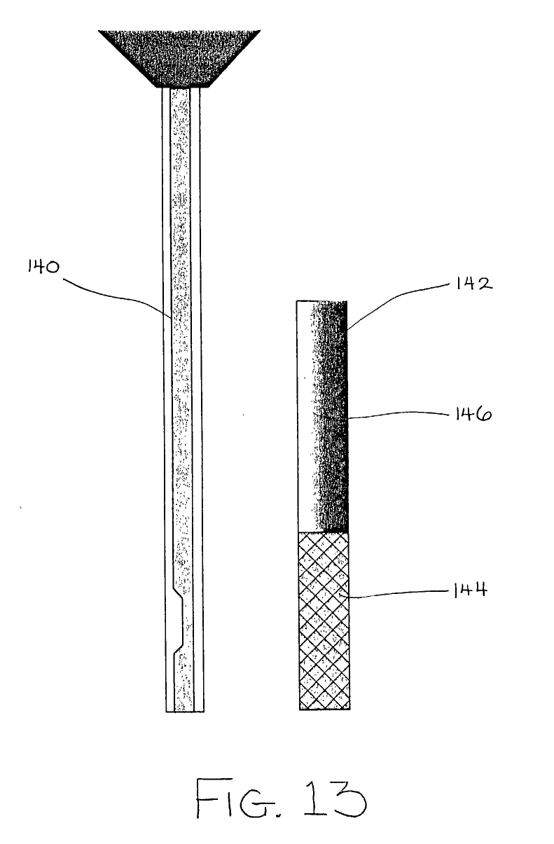

Reference is now made to FIGS. 11 and 12 of the drawings. FIG. 11 shows the biopsy needle 100, as well as the needle sheath 102. In use, as shown in FIGS. 12B to 12I, the sheath 102 surrounds and accommodates the needle 100, which is able to slide up and down within the hollow tube defined by the sheath. In FIGS. 12B to 12I, the sequential steps for the biopsy, and cauterizing of the tract formed by the removal of tissue from the organ illustrated, is described and shown in diagrammatic fashion. FIG. 12B shows the biopsy needle with the insulation thereabout. In FIG. 12C, the biopsy needle is placed at the surface of the skin 104, with the sheath pulled upwardly over the needle. In FIG. 12D, the needle is inserted by puncturing through the skin 104, passing through subcutaneous fat 106, tissues, muscle, and fascia, and just entering the organ. In FIG. 12E, the biopsy needle Third Sheath 108 is forced down around the needle, so that it too penetrates the skin and fat, and touches the organ 110 from which the tissue sample is being derived. FIG. 12F shows the following step in the biopsy, whereby the biopsy needle is now forced into the organ, by discharge thereof, and a sample of tissue 112 from the organ is captured within the needle in a recessed area 114 defined therein.

In FIG. 12G, the biopsy needle sheath is forced down partially into the organ. It will be noted that the biopsy needle sheath comprises a lower exposed portion 108, a middle insulated portion 116, and an upper portion 118 which is also exposed. In FIG. 12G, it will be noted that the lower exposed portion 108 is formed within the organ 110, with the insulated portion 116 extending from the surface of the organ to outside of the skin.

In FIG. 12H, an electro-cautery source 120, or a cryo-source, is connected to the upper exposed portion 118 of the sheath 102. At the same time, the needle 100 is withdrawn with the tissue sample 112 therein, as shown in FIG. 12I. While the needle is withdrawn, the outer sheath 102 remains unmoved. The application of the electro-cautery source 120, or the cryo-source, to the upper exposed portion 118 of the sheath 102 results in the lower exposed portion 108 within the organ 110 being substantially heated by the power, or frozen by the cryo-source, thereby hardening and/or obliterating and in this way plugging exposed vessels and other blood sources, to thus cauterize the tract. FIG. 12J shows the removal of the sheath 102, with the tract 124 duly cauterized to prevent bleeding.

FIG. 12I* shows an alternative arrangement whereby a liquid hemostatic agent 126, such as fibrin glue, is contained within a syringe, or other appropriate container 128, and inserted into the hollow sheath 102. When the syringe or other container reaches the lower unexposed portion, the syringe is punctured, or treated such that the hemostatic agent contained therein is released. The hemostatic agent has the effect of cauterizing the tract 124 to prevent or minimize bleeding.

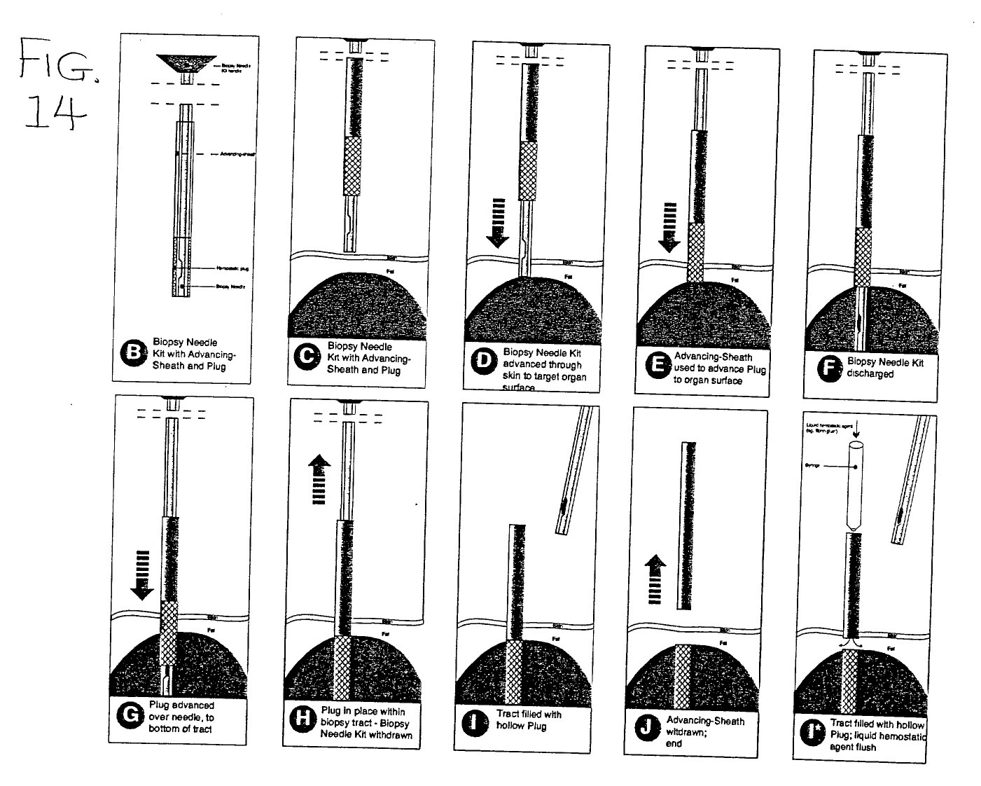

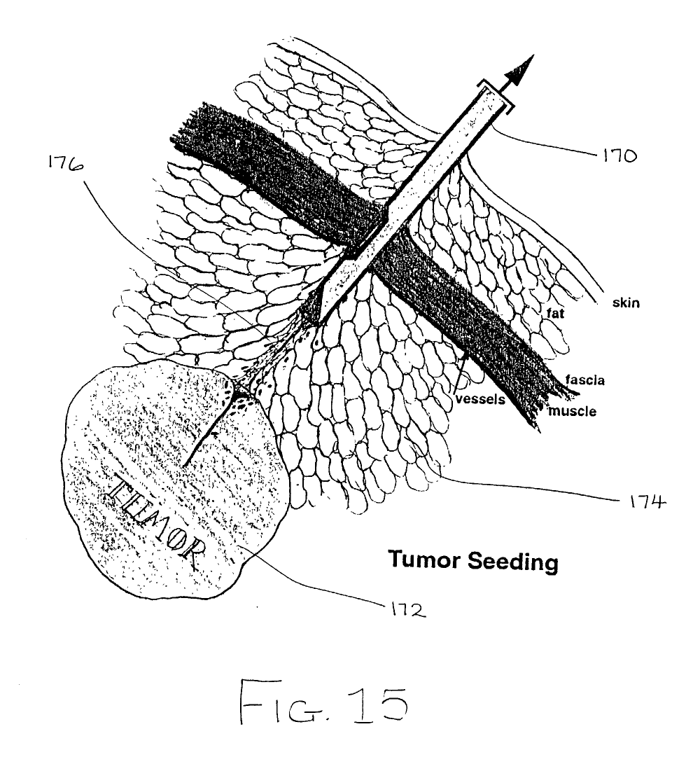

Reference is now made to FIGS. 13 and 14 of the drawings. FIG. 13 shows a larger diagrammatic version of the biopsy needle 140, with, at its side, the biopsy needle sheath 142 which will contain the needle 142. The sheath 142 comprises two distinct portions, the detachable or deployable plug 144, and the “Advancing Sheath” 146 which is used to push the deployable plug 144 into place and through which the biopsy needle 140 with specimen are withdrawn from the body. When both are slid over (loaded onto) the biopsy needle, elements 144 and 146 are contiguous.

FIG. 14B shows the biopsy needle 140 contained and slidable within the needle sheath 142. In FIG. 14C, the needle 140 is at the surface of the skin 104, with the sheath 142 pulled back, while in FIG. 14D, the needle 140 penetrates the skin 104 and is advanced to the target organ 110 surface, again with the sheath 142 pulled back. In FIG. 14E, the sheath 142 is pushed into the body, around the needle 140, so that both the needle 140 and the sheath 142 are at the surface of the organ 110. In FIG. 14F, the biopsy needle 140 is discharged, forcing it into the organ 110, and capturing a tissue sample 112 in a recess 114 within the needle 140.

In FIG. 14G, the outer needle sheath 142 is forced in around the biopsy needle 140, and advanced over the needle 140 so that, as shown in FIG. 14H, the plug portion 144 of the sheath 142 is in place within the biopsy tract 124. The biopsy needle 140 is then withdrawn, leaving the tract 124 filled with the hollow plug 144, as shown in FIG. 14I. In FIG. 14J the hollow plug 144 of the sheath 142 is detached from the remainder 146, with the remaining part 146 of the sheath 142 being withdrawn from the body. The plug 144 is left in place, as shown in FIG. 14J, plugging up sites and prevents further bleeding indirectly by plugging-up the site, and directly by promoting hemostasis by virtue of its hemostasis promoting material composition.

FIG. 14I* shows an alternative whereby a syringe 128 containing a hemostatic agent 126, such as “fibrin glue”, is inserted into the hollow tube, and released, when within the tube, so as to prevent bleeding. This is similar to the equivalent embodiment described in FIG. 12.

Further Description, Operation and Materials

A. Hemostasis by Cautery of the Biopsy Tract:

A metal sleeve is provided of diameter sufficient to closely fit over existing biopsy needles, while allowing it to be slid freely over and along the length of the existing biopsy needle. The length of the metal sleeve is less than the entire length of the biopsy needle, such that, once fitted onto the biopsy needle, it is flush with the proximal end of the biopsy needle (where-the biopsy needle junctures with the large “discharge unit” of the biopsy needle kit). The ends of the metal sleeve are preferably rounded, in contrast to the sharp-pointed end of the needle kit).

The metal sleeve is preferably insulated. The length of the metal sleeve, proximal to distal, can be divided for purposes of description, into three parts. Part A is approximately 25 mm long and un-insulated. Part B, from the end of part A to the beginning of part C, is insulated. The insulation material can be any commonly used medical-grade inert, non-toxic plastic material that is firmly adhered to the metal Third Sheath. The metal sleeve or third sheath, along the insulated length of part C, will not conduct electrical charge to the surrounding tissues it traverses. Part C, the distal tip of metal sleeve, has a length which may be equal to the biopsy tract length (“biopsy tract depth into target organ”) as determined by the manufacturer of the specific biopsy needle kit being used. For example, C. R. Bard, Inc. manufactures two different biopsy needle kits (16 Gauge and 18 Gauge needles) that both create a 22 mm tract length. The metal sheath of the invention designed to be used in conjunction with this company's needle specifications would therefore have an insulated tip length of 22 mm.

The sheath of the invention can be made to accommodate various manufacturers specifications regarding biopsy needle diameter (for snug but sufficiently loose fit to allow easy sliding of sheath over needle) and un-insulated tip length.

In operation, the metal sleeve is fitted onto the biopsy needle kit prior to biopsy. The metal sleeve is approximately the same length as the biopsy needle sufficiently long to span a minimum 5-10 +cm. from outside the body, down through subcutaneous tissues, to reach the bottom of the biopsy tract within the target organ. In other words, it is as approximately as long as the biopsy needle itself.

Electrical Current for cautery can be supplied either by touching the non-insulated proximal end of the sleeve with a conventional “Bovie Knife” tip (a Bovie Knife is a metal-tipped instrument that delivers a focus of cauterizing electrical current onto and through an electrically grounded patient). When a Bovie Knife tip touches the exposed, non-insulated conducting end of the metal sleeve, current is conducted along the length of the metal sleeve, through the insulated length, and to the non-insulated tip of the sleeve, which is snugly fit in the biopsy tract. The current cauterizes the tract.

Because performing needle biopsy in the outpatient setting is a closed procedure, the cauterization of the tract is not accomplished under direct visualization. Therefore, the user has no idea how either how long to apply the cautery power source, or what settings (voltage/current) are sufficient to achieve hemostasis. With such variables, potential operator error is a concern that can easily be addressed. For example, a “black box” interposed between the cauterizing metal sleeve and the power source may regulate the power applied to the sleeve such that only a standardized pre-set pulse reaches the metal sleeve for delivery to the organ biopsy tract.

Pulse settings (voltage/current) would be specific for both the biopsy needle kit for which the metal sleeve accessory is designed to be used, as well as for the specific target organ (i.e. with respect to tissue quality for cautery, and the particular organs' known propensity for bleeding). As an example, a specific manufacturers' biopsy needle kit has a needle diameter of X gauge and creates a biopsy tract of Y mm. A cauterizing metal sleeve is marketed to accommodate those particular specifications. The “black box” corresponding to this specific cauterizing metal sleeve is set to deliver, upon activation, one pulse of exactly the right strength (voltage) and duration (current) to achieve satisfactory hemostasis in the particular organ being biopsied. The exact settings are based on experimental data using live animal models for testing on various target organs. Presumably one organ type (e.g. liver) may require the application of more cautery when compared to a kidney.

The “black box” can be:

(1) A fuse-containing adapter that can be connected to the exposed end of the sleeve. Thus, any power source can be used, such that the adapter can be plugged into any Bovie cautery knife per source. Alternatively, the adapter can have an exposed metal conducting post such that a Bovie knife can itself be applied directly to the adapter.

(2) Alternatively, if no Bovie knife power unit is available in the particular use setting, a battery pack with a built-in fuse mechanism can be marketed for use with the cauterizing metal sleeve. It can be connected to the metal sleeve by conducting wires, etc. It can be mounted (“fit”) onto the handle of the needle biopsy apparatus, or, it can be freestanding, and rest on a table next to the patient, etc. This battery pack is switch activated to deliver a pre-set pulse, as discussed above. It contains sufficient charge to be fired multiple times either in the same patient or on multiple patients.

After cautery of the biopsy tract, the metal sleeve is withdrawn and discarded or sterilized, when appropriate.

B. Hemostasis of Biopsy Tract by Means of Filling Tract with a Hollow Hemostatic Plug

This aspect of the invention may consist of two pieces, together to be used in conjunction with any conventional needle biopsy kit as described below.

The first piece (piece #1) is a plastic semi-rigid inert plastic tube of only slightly larger diameter than biopsy needle. It is fitted over biopsy needle sufficiently loosely that it can be slid along the length of the biopsy needle. Its length is approximately ⅔ length of biopsy needle.

The second piece (piece #2), like piece #1 and of approximately equal diameter, is a thin-walled hollow flat-ended tube that fits over the biopsy needle. Two important features of this second piece are: 1. Length which equals the biopsy tract length as specified by particular biopsy needle kit manufacturer; and 2. Composition: It is made of a hemostatic material that is fully absorbable by the human body and can be left in place indefinitely. It can be essentially any currently used hemostatic material which lends itself to being manufactured in a tightly pressed form such that it can be made in the shape of a thin hollow tube to be fitted over the distal end of the biopsy needle kit. Its ends are rounded.

A variety of fully bio-absorbable topical hemostatic agents are currently marketed for use in surgical procedures. Each is made of a material(s) that are substrates of natural hemostatic pathways/reactions. These include Gelfoam™ (a dry spongy material made of processed collagen, which is activated prior to use by soaking in water) and Surgicel™ (also a cellulose product). Another such product is Ativene™, a collagen product that is used in a similar fashion for hemostasis. Either of these products could be used if they were to be pressed sufficiently to be no more than 1-2 mm thick, and if they could be made to, in the pressed tube shape, be more rigid. For example, the pressed tube form of the latter (or whatever hemostatic material is used) can be coated with processed collagen (itself fully bioabsorbable) to lend it the necessary firmness and smoothness to effortlessly be slid over the biopsy needle and into the biopsied organ.

In operation, pieces #1 and #2 are slid over the biopsy needle before use. Piece #1 is fitted over the needle first so that it extends to the proximal-most end of the biopsy needle (e.g. up to the biopsy needle kit handle.) Piece #2 is then also fitted over the biopsy needle end, and abuts piece #1. It is short (˜22 mm), and because it, like piece #1, is loaded over the biopsy needle as far proximally over the biopsy needle as it can go, its distal end is at least 20-50 mm. from the needle tip prior to firing the biopsy needle.

The biopsy needle, ensheathed by pieces #1 and #2 as above, is advanced under radiographic guidance from outside to in through the patient's skin, through subcutaneous tissue planes, to the surface of the target organ. The needle kit is then discharged, and, after discharge, the tip lies advanced deep into target organ, to depth =“biopsy tract length”.

After the needle kit discharge/biopsy, only piece #1 is visible extending out from the patient. Again, it is only semi-rigid, and inert. Piece #1 is held between the operators' thumb and index finger, and it is advanced gently towards needle tip, deeper into the patient along the length of the biopsy needle. (How far it is advanced can be made clear to the operator by, for example, imprinted colored arrows, etc, on piece #1 and the biopsy needle shaft.) In this way, piece #1 is used to advance piece #2 into the biopsy tract over a length exactly equal to the needle manufacturer's designated intra-organ biopsy tract length.

Once piece #1 is advanced into biopsy tract, the biopsy needle can be withdrawn from the patient.

When obtaining needle biopsy of tumor or potentially malignant tissue, and when the proposed accessory devices are being employed for purposes of limiting/preventing seeding of the needle's trajectory to the tumor with malignant cells, the device design can be modified to accentuate and further this purpose. For example, the Plug portion can be made slightly longer so that it extends outside of the visible organ/tumor margins.

Lengthening the Plug length serves the purpose of thereby limiting how close to the organ/tumor the Advancing Sheath has to approach. By eliminating or minimizing the contact that the Advancing Sheath has with the area of malignancy, the risk increases that, upon withdrawal, the advancing sheath will seed tumor/malignant cells to the needle tract lying between the biopsy target and the surface of the skin. Note: the Plug, which is contaminated with tumor cells, is never withdrawn in any way and remains in-situ after the procedure. Thus, the withdrawal of the biopsy needle and Advancing Sheath represents a “clean withdrawal”.

Furthermore, the Plug shape can be such that its proximal end “collapses” or tapers after withdrawal so as to limit tumor cells seeding escaping from the tumor site to the needle tract outside of the target area.

If necessary, the Plug can be coated with collagen to allow it to remain sufficiently rigid for deployment. Furthermore, the Plug could be impregnated with any number of chemotherapeutic/or other agents that either kill specific tumor cells/malignant cells, or otherwise prevent the migration of such cells. The Advancing Sheath could likewise be coated with a layer of such drug agents. Of note is that such drug agents would never risk contamination of the biopsy specimen because the biopsy needle discharges to obtain the biopsy specimen from tissue that lies ahead of (distal to) the drug impregnated plug and or Advancing Sheath. FIG. 14 shows how the Advancing Sheath and Plug initially lie far proximal to the needle tip are not advanced over the biopsy needle tip until well after the biopsy specimen is obtained.

In some scenarios, there might not be a clearly defined “biopsy target”, yet the entire field that the needle kit passes through might be considered “potentially malignant”. In such cases, where there is very high risk of seeding the needle's trajectory tract with tumor cells, one could employ either an even longer Plug (such that the proximal end of the Plug terminates in a known non-tumor cell containing field), or, the physician could employ the cauterizing “Third Sheath with Cautery” embodiment of the proposed accessory devices (shown in FIG. 12.) for purposes of exposing the entire needle trajectory to a pulse of cauterizing energy such that any “film” of tumor cells that were inadvertently seeded would be killed and thus neutralized. The physician could choose how much of the trajectory is desired and/or safe for cautery by simply adjusting the length of exposed (non-insulated) needle tip. A movable/adjustable insulating sheath, versus insulation affixed the Third Sheath previously described, would facilitate use for such purposes. The insulating sheath could be labeled in some way such that the physician could, with Radiographic input, determine the distance of the insulating sheath from the biopsy needle tip in order to help him/her visualize which tissues and what length of the needle trajectory will be cauterized.

After the biopsy needle is withdrawn, all that remains in place is piece #1 flush/contiguous with piece #2. Here, piece #1 can be pulled out and discarded, leaving behind piece #2, now inside the biopsy tract, yielding hemostasis.

Alternatively, prior to withdrawing and discarding piece #1, the continuity between pieces #1 and #2 can be capitalized upon. As an optional “extra” step, a syringe can be fitted onto the exposed open end of piece #1 and flushed with either fluid hemostatic agent (e.g. “Fibrin Glue”) or a solid hemostatic plug (morselized Gelfoam or Surgicel or other solid hemostatic material). This latter “extra step” can be taken as needed, for example, when performing biopsy of a target at particular risk for bleeding. Or, if, for example, bleeding is caused as the biopsy needle is advanced through the skin to the target organ (i.e. in the subcutaneous space), piece #1 can be withdrawn out of the target organ so that its tip lies in the subcutaneous space, and then it can be flushed with liquid hemostatic agent or solid hemostatic plug.

The surgical-steel third sheath, covered along a fraction of its length with a thin plastic electrical insulation covering, may be attached to either a electrical source (for cautery) OR a cryo source (for cryo “freezing” of the bleeding tract. The term “cryo-source” can refer to a source of extreme low temperature material such as, for example, liquid nitrogen, to which the sheath is coupled for purposes of achieving hemostasis within the tract. When using a cryo-source, the “insulating material” used on the sheath would be chosen for its ability to insulate surrounding tissue from excess cold.

FIGS. 13 and 14 “I*” illustrates the reference made above to using a liquid hemostatic material. The use of liquid hemostatic material is designed to be itself inserted into a bleeding tract for purposes of flushing the tract with the liquid hemostatic agent. The sheath of the invention seeks to capitalize on the hollow continuity with the biopsy tract created by either the cautery or the deployable hemostatic plug. In both cases, after the cautery or deployment of plug has occurred, the user has the option of flushing the aforementioned hollow conduit with a liquid hemostatic agent, as an “extra” measure. After flushing with the agent, the conduit is withdrawn, as usual.

Materials: The sheath may be of surgical steel, so as to conduct electrical current. The insulating Material may be thin coating (<1 mm) of plastic coating known to be and used in the Trade (surgical setting) as an electrical insulator.

The advancing Sheath may be a chemically inert, plastic, flexible tube that is firm enough to be able to (when pushed along its axis) push the deployable hemostatic plug through all tissue layers from the skin surface down through to the organ-biopsy tract. It can be made of anything plastic, firm and flexible.

The hemostatic plug may be a thin (˜1 mm.) pressed version of existing solid textured hemostatic agents used in the operating room, whereby these agents are laid down upon an open site of bleeding. These agents are fully absorbable, and act to stop bleeding by being impregnated with (or made of) natural components of the blood-clot forming pathway (e.g. Fibrin), or, substrate known to locally facilitate hemostasis (e.g. Collagen).

The liquid Hemostatic Agent: There are currently liquid hemostatic agents on the market designed to be sprayed onto or flushed into open sites of bleeding on the operating-room table. (e.g. “Fibrin Glue”).

For deep-tissue biopsy, where a longer biopsy needle and advancing sheath/plug apparatus are necessary, in order to prevent the advancing sheath from dissociating from the plug unit once the rigid biopsy needle has been withdrawn (see FIG. 14I), the advancing sheath can be designed such that it is slightly tapered at its distal end, so that it can fit into the proximal end of the plug. In this way, once the biopsy needle is withdrawn, the advancing sheath/plug continuity will be able to withstand bending at their meeting point without either separating or allowing a gap through which tumor cells could potentially be exposed to the surrounding tissues.

Prevention of Tumor Seeding

For the prevention of tumor seeding, as briefly discussed above, the sheath or bioabsorbable sheath of the invention serves, in its most basic form (i.e. where the bioabsorbable material contains no added auxiliary materials) to act as a “plug” that fills the defect within the tumor (caused by the needle and now absent tissue specimen) upon tumescence with moisture saturation. This hollow-turned “solid” gelatin plug may directly prevent or obstruct the leakage of microscopic tumor cells out of the tumor with blood and/or serous effluent.

In one aspect of the invention, before the bioabsorbable sheath has had the opportunity to become saturated, the biopsy needle with specimen are preferably withdrawn from the patient. The continuity of the “bioabsorbable sheath” flush with the “Advancing Sheath” creates a closed inner environment through which the biopsy needle tip passes. If the biopsy needle tip is considered “contaminated” with tumor material, the withdrawal of the biopsy needle thus occurs through this closed continuity of the “bioabsorbable sheath” and the “advancing sheath”, thus preventing the biopsy needle tip from making direct contact with any tissues, upon withdrawal, between the biopsy site and the skin. This can be referred to as the “clean withdrawal” (of the tumor cell-contaminated biopsy needle tip) feature of the invention. By preventing direct contact between the biopsy needle tip and superficial tissues, one method of tissue tract seeding is prevented.

It is preferable that the bioabsorbable sheath be made longer than the biopsy site depth-length so that the distal end of the Advancing Sheath does not have to make direct contact with the tumor (thus risking contamination). This is because the Advancing Sheath is the only element of the device that is withdrawn out to the skin through superficial tissue layers. Again, if the Bioabsorbable Sheath is made sufficiently long that its proximal end is proximal to the surface of the tumor, the Advancing Sheath, which lies flush to the proximal end of the Bioabsorbable Sheath, will not make direct contact with the tumor, and can be assumed to be “clean”. This is illustrated in the attached figures.

The distal advancement of the Advancing Sheath/Bioabsorbable Sheath unit can, in one preferred embodiment, be rendered a single handed procedure by incorporating an “Advancing Arm” on the handle of the biopsy needle kit. When a button on the biopsy needle handle is depressed, the “Advancing handle” is advanced distally over a pre-set distance. This preset distance can equal the distance necessary to advance the distal tip of the bioabsorbable sheath to the distal most tip of the biopsy needle. In this way, hand guidance of the biopsy needle, firing of the biopsy needle, and one-step push-button advancement of the bioabsorbable sheath exactly or accurately into place (calibrated for pre-set distance) can be achieved, as illustrated in the drawings.

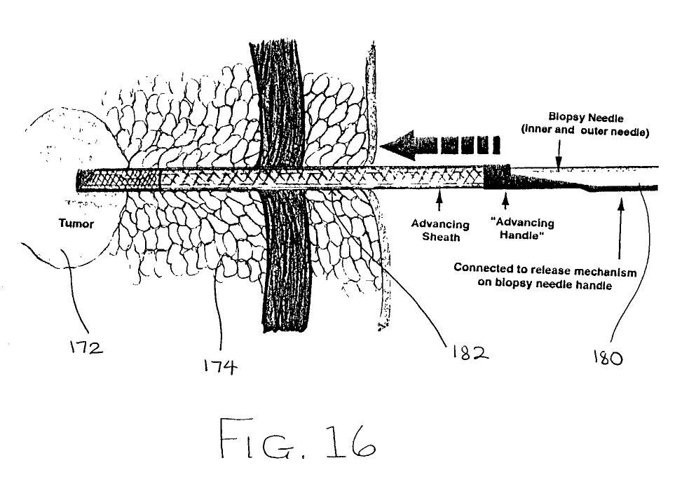

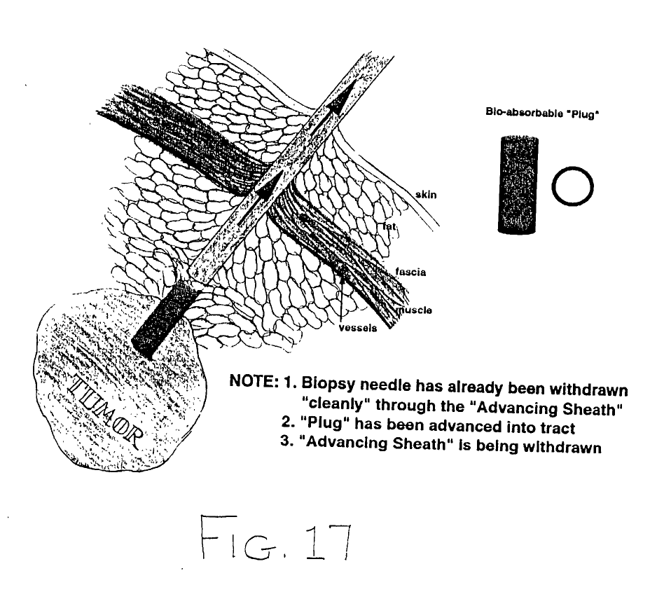

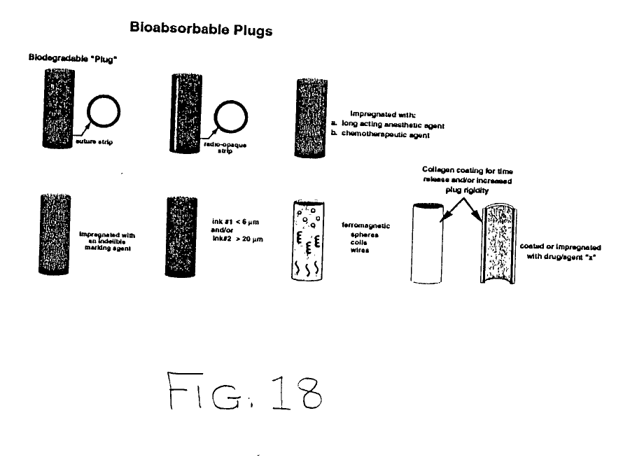

FIG. 15 shows tumor seeding which may be caused by conventional methods of biopsy. A biopsy needle 170 withdrawn from a tumor site 172 is dragged through intervening tissue 174 leaving cells or seeds therein. FIGS. 16 and 17 illustrate biopsy procedures using a biopsy needle 180 and sheath 182 in accordance with various aspects and embodiments of the invention. In FIG. 16, the biopsy needle 180 and sheath 182 in which it is contained are shown inserted in the tissue 174 and tumor site 172. Withdrawal of the needle 180 occurs wholly within and through the sheath 182 and no part of the needle 180 comes into contact with tissue 174. As such, no seeding of tumor cells can take place. FIG. 17 shows the stage where the needle 180 has been withdrawn and the sheath 182 is still in place, surrounding the entire route of withdrawal of the needle 180. The sheath 182 may be removed, or partially removed, so the tract is plugged, in accordance with the various embodiments and modifications described above.

A variety of materials can be incorporated into or onto the bioabsorbable sheath of the invention, as illustrated in the drawings. In this way, as described already, the device of the invention can also serve as a platform for the delivery of various diagnostic and therapeutic materials into the target site. An example of some of the main optional materials that can be incorporated into the bioabsorbable sheath are set out below, although the invention is not limited to these materials.

(A) Indelible ink. This is useful to visibly mark the biopsy tract so that, upon subsequent open surgical procedure, the entire tract can be excised, as is currently recommended with biopsy of many malignant masses. Any ink with ink particles approximately >20 micrometers in diameter will unlikely be able to be processed or engulfed (and thus removed) by local phagocytic cells.

(B) An ink (or radioactive colloid) agent that is easily taken-up by local lymphatics. Ink particles approximately <8 micrometers can easily be taken up into local lymphatics, thus serving as a “sentinel lymph node marking device” simultaneous to needle biopsy, and prevent hemorrhage/tumor seeding. This is of unique benefit because the introduction of sentinel lymph node marking dye is done by separate needle injection after biopsy. This is sub-optimal for two reasons. First, it requires a two-step procedure, which is time consuming, increases pain or discomfort to the patient, and necessitates anesthesia (which has its own inherent risks) more than once. Second, such a two-step procedure has less specificity for true sentinel lymph node identification, because it is always possible (and likely) that the marking agent will not be injected into the exact site that was biopsied. This disconnect could alter treatment decisions that could render occult metastases undetected, untreated, and/or unresected. This is potentially harmful because the operator has no way of confirming whether the injected marker was indeed placed at the exact site of biopsy.

Any given bioabsorbable sheath could contain (in addition to other materials) both the low particle diameter marking agent (for lymphatic uptake for “Sentinel Lymph Node marking”), as well as the high particle diameter agent (for indelible plainly-visible marking of the biopsy tract).

(C) The Bioabsorbable Sheath could contain any drug or chemical agent, as desired. It could even contain such a drug agent within a time-release matrix, to allow for slow release of the drug, as desired.

(D) The Bioabsorbable Sheath could contain any type of ferromagnetic element(s) which themselves can later be “activated”, by radio-frequency signal, to deliver a highly focused dose of thermo-ablation to the tumor/tissue target. Such ferromagnetic elements can take any shape, including sphere's, straight wires, and/or coils.

(E) The Bioabsorbable Sheath can contain a non-absorbable, safe, non-toxic “marker element” which is easily visualized at open surgical procedure. This material may comprise, for example, a small piece of brightly colored nylon suture, clip, sphere, etc.

(F) The Bioabsorbable Sheath can include any material element which is to be implanted deep within or at the target tissue site, and is to extend therefrom to the outside of the patient.

(G) The Bioabsorbable Sheath can be coated with a collagen (or otherwise firm, non-toxic bioabsorbable material coating) to lend added firmness to the bioabsorbable sheath (for example, in cases where it will be used in particularly tough-to-penetrate tissues), and/or, to delay the time before tumescence of the bioabsorbable sheath.

In FIG. 18 of the drawings, there are shown a variety of different types of plugs or the material that they may carry. These bioabsorbable or biodegradable plugs may include: a suture strip, a radio-opaque strip, impregnation with anesthetic agent or chemotherapeutic agent, impregnation with an indelible marking agent, two or more ink types, ferromagnetic material, and/or collagen coating for time release and/or increased plug rigidity. These are examples only, and other materials or compositions may be carried by the plug according to specific needs. Further, combinations of materials and compositions may also be carried, as needed.

While the sheath of the invention in its various forms and embodiments allows for use as a biopsy-needle accessory device for hemostasis (both the “cauterizing over-sheath” and the “bioabsorbable sheath” devices), and for prevention of tumor seeding, the overall design of the “bioabsorbable sheath” device of the invention can serve as a “materials delivery device” to be used with any biopsy needle system, as well as any system that introduces a long, tube-like structure into the body. The sheath of the invention is something can be introduced over the tube (tube, biopsy needle, etc) being inserted into the body.

Another important feature of the device of the invention is that any such material(s) can be thus delivered within a “protected” delivery vector material such as any currently used bioabsorbable surgical hemostatic materials (e.g. Gelfoam™, a sterile, water insoluble, malleable, bioabsorbable porcine-derived gelatin manufactured by Pharmacia and Upjohn, or Surgicel™, an oxidized regenerated cellulose product with similar properties, manufactured by Johnson and Johnson). Such products have heretofore been used topically in open surgery generally only for hemostasis. The present invention facilitates that these products (or similar products that are sterile, water insoluble, malleable, and most importantly, bioabsorbable) can be used to contain and deploy materials deep into spaces within the body through the use of a minimally invasive device such as a biopsy needle (or similar “tube”, probe, etc, inserted in a minimally-invasive technique). The invention thus allows for a method by which materials can thus be delivered into the body, in a single step, minimally invasive technique.

The invention provides and effective device and procedure whereby bleeding can be controlled during biopsy proceedings. While a number of embodiments and forms of the invention have been discussed, it will be appreciated that the invention is not to be limited to the precise details described above.

1. A sheath for a biopsy needle for removing a sample from a living body, the sheath comprising:
a substantially tubular structure configured to accommodate at least a portion of a biopsy needle, the tubular structure having a side wall, a first end opening which in use will remain outside of the body and a second end opening which will in use insert into the body, the tubular portion having a length between the first and second openings which is greater than the distance between sample to be removed and the outside of the body.

2. A sheath as claimed in claim 1 wherein the sheath comprises a treating segment extending from second opening for treating tissue adjacent thereto when the sheath is located in the body.

3. A sheath as claimed in claim 2 wherein the treating segment comprises a cauterizing segment which is heated by an external source.

4. A sheath as claimed in claim 2 wherein the treating segment comprises a cooling or cryo segment which is cooled by an external source.

5. A sheath as claimed in claim 1 comprising a conductive tubular structure having a centrally located insulating material thereabout so as to define upper and lower exposed conductive portions of the tubular structure, the upper conductive portion being connectable to a treatment source to supply energy, the centrally located insulating material facilitating handling of the sheath by a user.

6. A sheath as claimed in claim 1 wherein the sheath comprises a detachable segment extending from the second opening and a body removable segment coaxial with the detachable segment, the detachable segment being selectively detachable to remain in the body after the removable segment has been removed to thereby plug an open tract corresponding to the sample.

7. A sheath as claimed in claim 6 wherein the detachable segment comprises a delivery vehicle for delivering a selected composition to the body.

8. A sheath as claimed in claim 7 wherein the selected composition is selected from one or more of the group consisting of: indelible ink, an ink agent, a radioactive colloid, a chemical or drug agent, a ferromagnetic element, and a non-toxic marker element.

9. A sheath as claimed in claim 7 wherein the detachable segment is comprised of a bioabsorbable or a biodegradable material.

10. A sheath as claimed in claim 1 further comprising a delivery vehicle for delivering a selected composition to the body.

11. A sheath as claimed in claim 1 wherein the tubular structure has leading edge at second opening which is pointed to facilitate easy entry and travel through the body to the sample site.

12. A biopsy needle and sheath kit comprising:
biopsy needle for removing a sample from a living body, the biopsy needle having an elongate needle having a distal end for extracting the sample from the body; and
a sheath comprising a substantially tubular structure configured to accommodate at least a portion of a elongate needle, the tubular structure having a side wall, a proximal end opening which in use will remain outside of the body and a distal end opening which will in use insert into the body, the tubular portion having a length between the proximal and distal openings which is greater than the distance between sample to be removed and the outside of the body, the sheath providing a passage for the elongate needle, the needle and sheath being coaxial and capable of independent axial movement relative to each other.

13. A delivery vehicle for introducing a predetermined composition to a living body, the delivery vehicle comprising a substantially tubular structure having a side wall, a first end opening which in use will remain outside of the body and a second end opening which will in use insert into the body, the tubular portion having a length between the first and second openings which is greater than the distance between sample to be removed and the outside of the body, the distal end having associated therewith the composition which is transferred to the body when inserted therein.

14. A delivery vehicle as claimed in claim 13 wherein the tubular structure has a detachable segment at its distal end and a remaining segment, the detachable segment selectively detaching from the remaining segment to remain in the body after the remaining segment has been withdrawn.

15. A delivery vehicle as claimed in claim 13 wherein the composition is selected from one or more of the group consisting of: indelible ink, an ink agent, a radioactive colloid, a chemical or drug agent, a ferromagnetic element, and a non-toxic marker element.
